(19) United States
(12) Patent Application Publication
(10) Pub. No.: US 2025/0082488 A1
(43) Pub. Date: Mar. 13, 2025

(54) MEDICAL SUPPORT BOOT WITH IMPROVED ANKLE AND HEEL PROTECTION

(71) Applicant: Sally Arrey, Lee's Summit, MO (US)

(72) Inventors: Sally Arrey, Lee's Summit, MO (US); Richard Chungong, Lee's Summit, MO (US)

(21) Appl. No.: 18/826,613

(22) Filed: Sep. 6, 2024

Related U.S. Application Data
(60) Provisional application No. 63/537,279, filed on Sep. 8, 2023.

(51) Int. Cl. A61F 5/01; A43C 11/08; A61F 13/06

(52) U.S. Cl. CPC A61F 5/0195; A43C 11/08; A61F 13/069

(57) ABSTRACT

A protective boot includes an insert having a first ankle portion and a second ankle portion. The first ankle portion includes a first ankle indentation shaped and positioned to receive a medial malleolus of a user when the protective boot is worn. Similarly, the second ankle portion includes a second ankle indentation shaped and positioned to receive a lateral malleolus of the user when the protective boot is worn. The insert further includes a plantar portion including a heel indentation (which may optionally extend through the insert to form a heel hole) shaped and positioned to receive a heel of the user when the protective boot is worn. The protective boot further includes a cover conforming to and enclosing the insert, the cover including an opening shaped to receive the insert and a fastener assembly for securing the protective boot to a foot of the user.

### CROSS-REFERENCE TO RELATED APPLICATIONS

This application claims the benefit of U.S. Provisional Patent Application No. 63/537,279, filed Sep. 8, 2023, which application is incorporated by reference in its entirety herein.

### FIELD

The present disclosure relates generally to a protective medical boot for supporting, shielding, and promoting the healing of a foot of a user or patient.

### BACKGROUND

Pressure injuries, such as pressure ulcers, are a common occurrence and problem for substantially immobilized individuals, such as patients that are post-surgery or that have health conditions causing them to be bedridden. Preventing such injuries generally includes offloading or raising the corresponding body part above the supporting surface.

The heel and ankles are common areas prone to development of pressure ulcers for immobile patients. Among other preventative measures, such ulcers can be prevented or mitigated with the help of heel protection devices or similar supporting “boots.” In general, such devices are worn on the foot to elevate the heel of the patient and provide padding between prominences of the foot (e.g., the heel or ankles) and the underlying supportive surface (e.g., the patient's bed). Doing so provides direct protection to the prominences while also shifting and redistributing the weight of the lower leg to larger areas of the patient's body, such as the lower thigh.

Conventional medical boot designs generally include an outer shell stuffed with a fill material, such as batting. In general, these conventional designs are large and bulky, often resembling a pillow strapped around the foot of the patient. The bulk material also often acts as insulation, causing the foot to become uncomfortable and generally discouraging prolonged use by the patient.

It is with the foregoing issues in mind, among others, that aspects of the disclosure were conceived and developed.

### SUMMARY

Briefly described, one embodiment of the present disclosure comprises a protective medical boot that includes an insert having a first ankle portion and a second ankle portion, with the first ankle portion including a first ankle indentation or pocket that is shaped and positioned to receive, support and protect a medial malleolus of a user when the protective boot is worn. Similarly, the second ankle portion includes a second ankle indentation or pocket that is shaped and positioned to receive, support and protect a lateral malleolus of the user when worn. The insert further includes a plantar portion including a heel indentation (which may optionally extend through the insert to form a heel hole) that is shaped and positioned to receive a heel of the user when the protective boot is worn. The insert is formed as a shaped layer of solid gel or gel-like flexible material. The protective boot further includes a cover that closely conforms to and encloses the insert, with the cover including an opening shaped to receive the insert and one or more fastener assemblies for securing the protective boot to a foot of the user.

In accordance with another embodiments, the protective boot further includes an Achilles support portion extending from the heel indentation or hole upwards between the first ankle portion and the second ankle portion, and which includes an Achilles support protrusion with a curved shape that generally conforms to the Achilles tendon of the user. The solid gel material forming the ankle indentations or pockets and the Achilles support protrusion can be continuous or can include a plurality of small openings or perforations to promote air circulation around the foot of the user.

Other additional embodiments of the present disclosure will be better understood upon review of the detailed description set forth below taken in conjunction with the accompanying drawing figures, which are briefly described as follows.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a plan view of a protective medical boot, in accordance with one representative embodiment or implementation of the present disclosure.

FIG. 2 is a cross-sectional view of the protective boot of FIG. 1.

FIG. 3 is a perspective view of the protective boot of FIG. 1 positioned on a foot of a patient prior to securing of the protective boot to the foot.

FIG. 4 is a perspective view of the protective boot of FIG. 1 secured to the foot of the patient.

FIG. 5 is an exploded view of the protective boot of FIG. 1.

FIG. 6 is a plan view of the medical protective boot, in accordance with another representative embodiment or implementation of the present disclosure.

FIG. 7 is a plan view of an insert of the protective boot of FIG. 6.

FIG. 8 is a cross-sectional view of the insert of FIG. 7.

FIG. 9 is a plan view of the medical protective boot, in accordance with yet another representative embodiment or implementation of the present disclosure.

FIG. 10 is a back-side perspective view of an insert of the protective boot of FIG. 9.

Those skilled in the art will appreciate and understand that, according to common practice, various features and elements of the drawings described above are not necessarily drawn to scale, and that the dimensions of the various features and elements may be expanded or reduced to more clearly illustrate the embodiments of the present disclosure described therein.

### DETAILED DESCRIPTION

The following description, in conjunction with the accompanying drawings described above, is provided as an enabling teaching of exemplary embodiments of a protective boot for shielding and promoting the healing of a foot of a user, and one or more methods for attached the protective boot the foot of the user. As described herein, the protective boot can provide several significant advantages and benefits over other conventional boot designs. However, the recited advantages are not meant to be limiting in any way, as one skilled in the art will appreciate that other advantages may also be realized upon practicing the present disclosure.

Furthermore, those skilled in the relevant art will recognize that changes can be made to the described embodiments while still obtaining the beneficial results. It will also be apparent that some of the advantages and benefits of the described embodiments can be obtained by selecting some of the features of the embodiments without utilizing other features, and that features from one embodiment may be combined with features from other embodiments in any appropriate combination. For example, any individual or collective features of method embodiments may be applied to product or system embodiments, and vice versa. Accordingly, those who work in the art will recognize that many modifications and adaptations to the embodiments described are possible and may even be desirable in certain circumstances, and are a part of the disclosure. Thus, the present disclosure is provided as an illustration of the principles of the embodiments and not in limitation thereof, since the scope of the invention is to be defined by the claims.

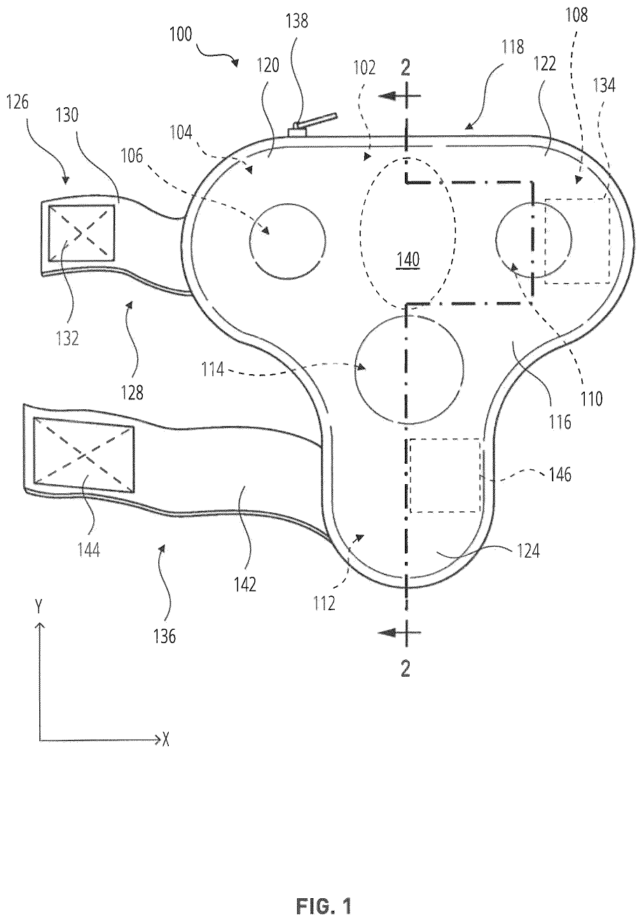

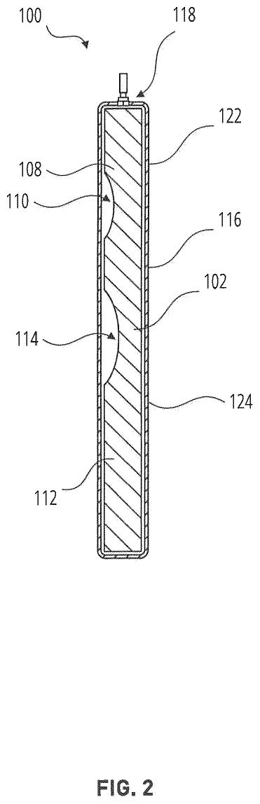

Referring now in more detail to the drawing figures, wherein like parts are identified with like reference numerals throughout the several views, FIG. 1 is a plan view of a protective medical boot 100 according to a representative embodiment or implementation of this disclosure. FIG. 2 is a cross-sectional view of protective boot 100 as viewed along section line 2-2, indicated in FIG. 1. As illustrated, protective boot 100 can include insert 102 disposed within and enclosed by a cover 116, with each having a generally T-shaped construction. More specifically, insert 102 can include first ankle portion 104 which extends in a first direction and a second ankle portion 108 which extends equal and opposite first ankle portion 104. Insert 102 can further include a plantar portion 112 projecting perpendicular to each of first ankle portion 104 and second ankle portion 108.

Insert 102 can be shaped to conform to a foot of a patient/user when worn. More specifically, first ankle portion 104 can include a first ankle indentation 106, second ankle portion 108 can include a second ankle indentation 110, and plantar portion 112 can include a heel indentation 114. As discussed below in further detail, when worn, the malleoli (commonly referred to as the ankle bones) can be received within first ankle indentation 106 and second ankle indentation 110 while the heel can be disposed within heel indentation 114.

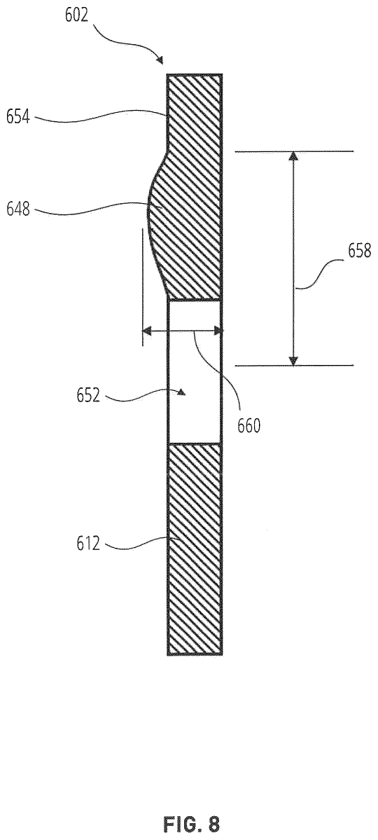

Insert 102 can be generally formed from a flexible material such that, when unworn, protective boot 100 has a substantially planar shape. Among other things, this notably reduces the space necessary to store and transport protective boot 100. Insert 102 can be formed from various materials; however, in at least certain implementations, insert 102 can be formed from a solid gel or gel-like flexible material, such as a medical grade silicone rubber gel, which has been formed into a shaped, substantially planar structure having a generally uniform thickness but with additional indentations, voids and/or protrusions included therein, as shown in FIGS. 2 and 8 and described in more detail below. In use, insert 102 can then be folded around the foot of the user, as also described below, to form a complex yet flexible three-dimensional body. When formed using silicone rubber gel or a similar material, insert 102 has relatively low insulative properties, thereby reducing potential overheating of the foot and improving patient comfort. Use of gel materials for insert 102 also permits insert 102 to be generally thinner as compared to conventional batting-filled protective boots without sacrificing support and protection provided by insert 102. Solid gel or gel-like flexible materials, such as silicone rubber gel, also have the benefit of readily conforming to patient anatomy and accordingly provide a form fit to the patient. Among other advantages, such a fit enables protective boot 100 to be substantially smaller and more streamlined than conventional designs without sacrificing robust support.

In certain conventional medical boot designs, indentations (such as first ankle indentation 106 and second ankle indentation 110) or similar features are omitted such that the conventional boot has a substantially uniform thickness. While such designs can provide adequate padding of the ankle and heel, the additional padding can often place the foot at an unnatural and uncomfortable angle relative to the rest of the patient's leg. Alternative conventional designs address this issue by including an holes or similar openings extending through the protective boot. In practice, however, such openings regularly permit at least the malleoli to contact the underlying supporting surface, allowing pressure and/or abrasive sores to develop on the patient's foot. In contrast to these conventional designs, which generally involve a tradeoff between foot positioning and foot protection, protective boot 100 provides both. More specifically, by forming insert 102 from the shaped layer of solid gel or gel-like flexible material, insert 102 can be made to have a relatively low thickness while providing adequate structural support to the patient's foot, with additional pressure-relieving clearances for the protruding bony structures of the foot provided by the indentations, voids and/or protrusions included therein. Stated differently, the use of the shaped layer of gel-like flexible material can permit the inclusion of first ankle indentation 106 and second ankle indentation 110, each of which can accommodate the shape of the ankle while simultaneously covering and protecting the malleoli to reduce pressure- and abrasion-related sores that can occur with open ankle boot designs.

Cover 116 can be sized and shaped to conform to insert 102. Accordingly, cover 116 can include a first ankle cover portion 120 containing first ankle portion 104 of insert 102, a second ankle cover portion 122 containing second ankle portion 108 of insert 102, and plantar cover portion 124 containing plantar portion 112 of insert 102.

As illustrated, cover 116 can include an opening 118 through which insert 102 may be inserted and removed from cover 116. Among other things, removal of insert 102 from cover 116 permits easy washing and maintenance of cover 116. To retain insert 102 within cover 116, cover 116 can include an optional closure 138. As shown, closure 138 can be in the form of a zipper; however, this disclosure contemplates the use of other closures such as, but not limited to a sliding channel closure, a hook-and-loop style closure, and a magnetic closure. As an alternative to including a positive closure mechanism, cover 116 may be formed from an elastic or semi-elastic material such that cover 116 tightly conforms to insert 102 and retains insert 102 by a friction fit.

As already noted, cover 116 may be formed from an elastic or semi-elastic material such that cover 116 may be stretched about insert 102. For example, cover 116 may be formed from a 4-way stretch material. In certain implementations, the material of cover 116 may also be moisture-wicking. Non-limiting examples of moisture-wicking materials suitable for use in cover 116 include polyester, polypropylene, nylon, micromodal rayon, and bamboo. In other implementations, the material of cover 116 may also or alternatively have quick-drying properties. Examples of such materials include, but are not limited to polyester and polyester-cotton blends. Cover 116 may also have anti-microbial properties, either through use of an anti-microbial material or by coating or similar application of an anti-microbial treatment.

As discussed below in further detail, when worn, a first malleolus of the ankle can be received in first ankle indentation 106, a second malleolus of the ankle can be received in second ankle indentation 110, and the patient's heel can be placed into heel indentation 114. Protective boot 100 can then be secured using a fastener assembly 126. In the specific implementation shown, fastener assembly 126 can include an ankle fastener 128 and a plantar fastener 136 for securing protective boot 100 to the patient's foot. In general, ankle fastener 128 can be configured to wrap each of first ankle portion 104 and second ankle portion 108 of insert 102 about the ankle of the patient. So, for example, ankle fastener 128 is shown in FIG. 1 as a strap coupled to an exterior surface of first ankle cover portion 120 and including a first portion of a hook-and-loop style fastener (e.g., a hook patch). When worn, the strap extends from first ankle cover portion 120 across a lower shin of the patient to couple to a second portion of the hook-and-loop style fastener (e.g., a loop patch) disposed on an exterior surface of second ankle cover portion 122. Similarly and generally speaking, plantar fastener 136 can be configured to retain plantar portion 112 against a sole of the patient's foot. So, for example, plantar fastener 136 is shown in FIG. 1 as a strap coupled to an exterior surface of plantar cover portion 124 and including a first portion of a hook-and-loop style fastener (e.g., a hook patch). When worn, the strap extends from the exterior surface of plantar cover portion 124 across an instep of the patient to couple to a second portion of the hook-and-loop style fastener (e.g., a loop patch) also disposed on the exterior surface of plantar cover portion 124.

As shown in FIG. 1, protective boot 100 can further include an Achilles support region 140 positioned between first ankle indentation 106 and second ankle indentation 110 (and above heel indentation 114). Due to the positioning of first ankle indentation 106, second ankle indentation 110, and heel indentation 114, when protective boot 100 is worn by a patient, the Achilles tendon and surrounding region can be contacted and supported by Achilles support region 140.

The overall size and dimensions of protective boot 100 and its components may vary to accommodate the anatomy of a range of patients. In general, accommodating a particular patient generally includes adjusting the overall dimensions of protective boot 100 to correspond to the general anatomy of the patient and/or modifying the size or placement of first ankle indentation 106, second ankle indentation 110, and heel indentation 114. In certain implementations, protective boot 100 may be offered in a range of fixed sizes (e.g., child, small, medium, large, etc.) with each size intended to fit a range of patient anatomies. In other implementations, protective boot 100 may be customized to fit a particular patient. For example, in one implementation, certain key dimensions (e.g., overall foot length, vertical distance between the heel and malleoli, etc.) may be obtained for a patient. Insert 102 may then be formed with first ankle indentation 106, second ankle indentation 110, and heel indentation 114 positioned based on the obtained measurements. In other implementations, insert 102 may be formed using a molding technique in which a mold of the patient's foot provides the general shape and dimensions of insert 102.

The dimensions of protective boot 100 may vary to accommodate the anatomy of different patients, however, in at least one specific example, protective boot 100 may have an overall width (from end-to-end across first ankle portion 104 and second ankle portion 108, excluding fastener assembly 126 and along the y-direction as indicated in FIG. 1) from and including about 10 inches to and including about 14 inches. For example, in one specific implementation, protective boot 100 may have an overall width of approximately 12 inches. Similarly, protective boot 100 may have an overall length (measured from a top of protective boot 100 to the extent of plantar portion 112 along the y-direction as indicated in FIG. 1) from and including about 10 inches to and including about 14 inches. For example, in one specific implementation, protective boot 100 may have an overall length of approximately 12 inches. With respect to the overall length of protective boot 100 (i.e., in the y-direction as indicated in FIG. 1), first ankle portion 104 and first ankle indentation 106 may extend from approximately one-quarter to approximately one-half to one-quarter of the total length of protective boot 100 while plantar portion 112 may extend the remaining length. In one specific example, first ankle portion 104 and first ankle indentation 106 may extend approximately one-third of the total length of protective boot 100 while plantar portion 112 may extend the remaining two-third of the total length of protective boot 100. So, for example, in one such example implementation, first ankle portion 104 and first ankle indentation 106 may extend approximately 4 inches in the y-direction while plantar portion 112 may extend approximately 8 inches in the y-direction. The width (i.e., in the x-direction) of plantar portion 112 may likewise vary; however, in certain implementations, plantar portion 112 may have a width from and including about 4 inches to and including about 8 inches, e.g., 6 inches.

As with the overall dimensions of protective boot 100, the dimensions and configuration of first ankle indentation 106, second ankle indentation 110, and heel indentation 114 may vary in implementations of this disclosure. However, in at least certain implementations, each of first ankle indentation 106 and second ankle indentation 110 may be generally circular in shape and have a diameter from and including about 1.25 inches to and including about 2.5 inches. For example, each of first ankle indentation 106 and second ankle indentation 110 may have a diameter of approximately 1.75 inches. Similarly, the depth of first ankle indentation 106 and second ankle indentation 110 may also vary. For example, in certain implementations, the depth of first ankle indentation 106 and second ankle indentation 110 relative to a planar top surface of insert 102 may be from and including about 0.5 inches to and including about 1.5 inches. For example, each of first ankle indentation 106 and second ankle indentation 110 may have a depth of approximately 1 inch. The overall thickness of insert 102 may vary in implementations of this disclosure; however, the thickness of insert 102 can generally be greater than the depth of first ankle indentation 106 and second ankle indentation 110 such that each of first ankle indentation 106 and second ankle indentation 110 is a closed pocket.

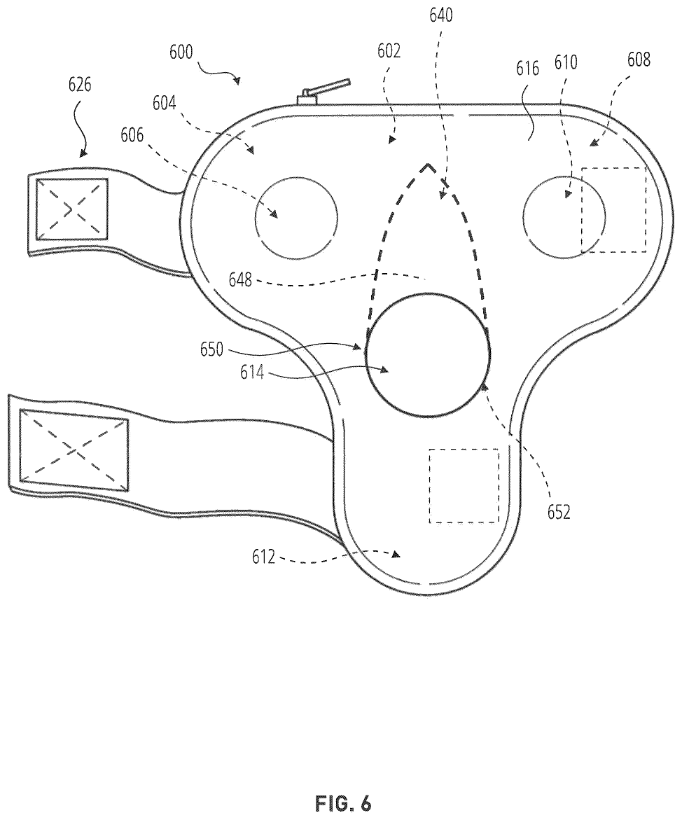

The depth of heel indentation 114 may be similar to that of first ankle indentation 106 and second ankle indentation 110 and less than the overall thickness of insert 102; however, in certain implementations, heel indentation 114 may extend fully through insert 102, thereby forming a through hole. An example of such an implementation is discussed below in further detail in the context of FIG. 6 to FIG. 8.

While certain example dimensions are provided throughout this disclosure, such dimensions are intended to be non-limiting. Stated differently, this disclosure contemplates that any dimensions noted in this disclosure may be readily modified (e.g., to accommodate a specific patient's anatomy) and still be within the scope of this disclosure.

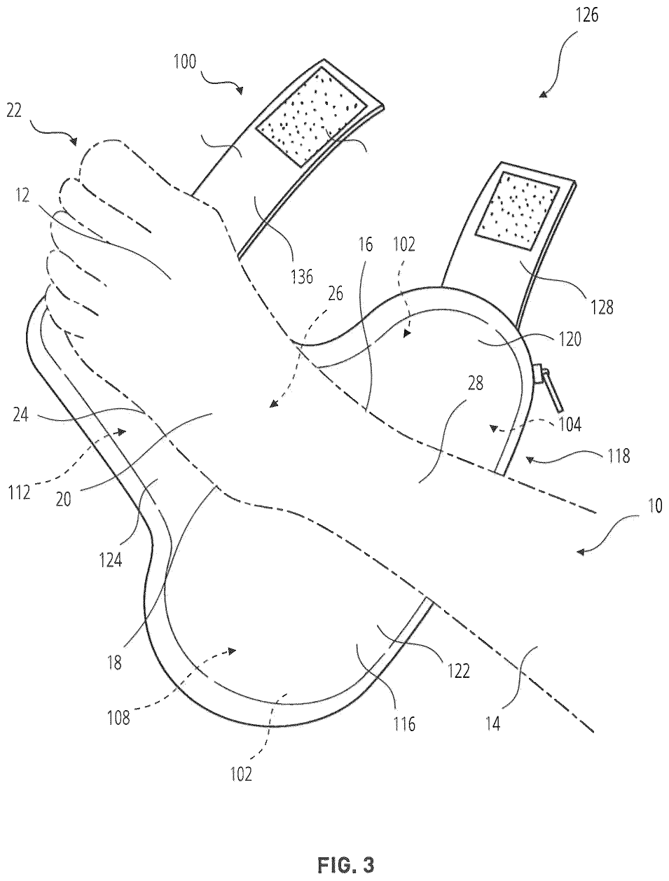

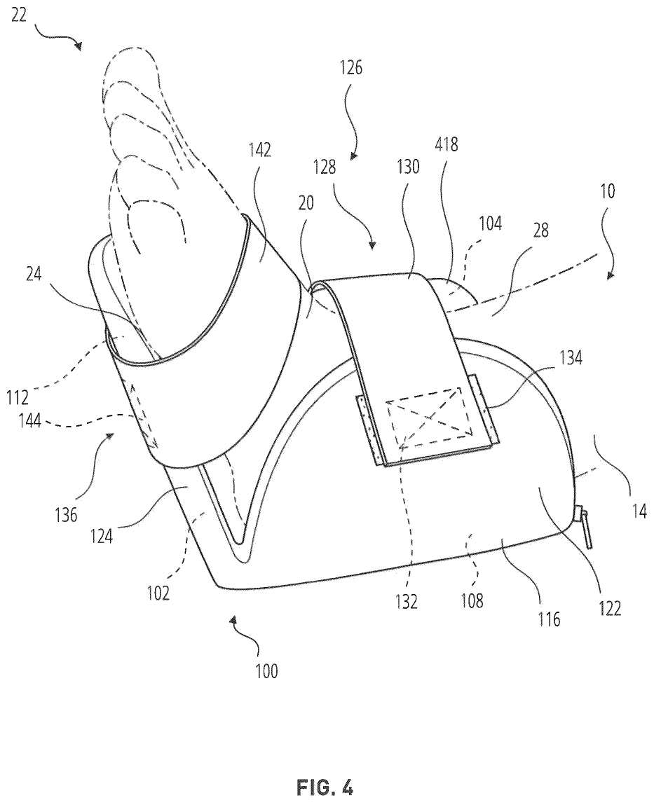

To further illustrate use of protective boot 100, FIG. 3 and FIG. 4 are illustrations of protective boot 100 in use by a patient 10. More specifically, FIG. 3 illustrates a foot 12 of patient 10 following placement of a heel 26 of patient 10 within heel indentation 114 but prior to engaging any of the fasteners of protective boot 100 to secure protective boot 100 to foot 12. As shown in FIG. 3, donning protective boot 100 generally includes first placing a heel 26 of patient 10 into heel indentation 114.

With heel 26 in position, patient 10 or an individual assisting patient 10 can secure plantar portion 112 or the ankle portions (i.e., first ankle portion 104 and second ankle portion 108) in either order. To secure plantar portion 112, plantar portion 112 can be folded up and against sole 24 of patient 10. Once in position, plantar fastener 136 can be secured. In the implementation shown in FIG. 3, plantar fastener 136 can include a plantar strap 142 coupled to an underside of plantar cover portion 124. To secure plantar fastener 136, the plantar strap 142 can be pulled across an instep 20 of patient 10 and secured to the underside of plantar cover portion 124 or at a similar location on an opposite side of foot 12. In the specific implementation shown, plantar fastener 136 can include a hook-and-loop style fastener to secure plantar strap 142. More specifically, plantar strap 142 ends in a plantar strap patch 144 including a first half of the hook-and-loop style fastener. When protective boot 100 is worn, plantar strap 142 extends across instep 20 and secures to a plantar cover patch 146 (shown in FIG. 1) attached to the underside of plantar cover portion 124 and which forms the second half of the hook-and-loop style fastener.

In the illustrated implementation, first ankle portion 104 and second ankle portion 108 secured similarly to plantar portion 112. To secure first ankle portion 104 and second ankle portion 108, are folded up against an ankle of patient 10. FIG. 3 and FIG. 4 illustrate protective boot 100 being worn on a left foot of patient 10 such that first ankle portion 104 can be placed against medial malleolus 16 of patient 10 while second ankle portion 108 is placed against lateral malleolus 18 of patient 10. More specifically, first ankle portion 104 can be positioned such that medial malleolus 16 is disposed within first ankle indentation 106 while second ankle portion 108 is positioned such that lateral malleolus 18 is disposed within second ankle indentation 110. Notably, in one aspect the implementation of protective boot 100 illustrated in the figures can be symmetrical, and as such intended to be wearable on either the left or right foot of a patient. Accordingly, when worn on the right foot, the lateral malleolus of the right foot can be disposed within first ankle indentation 106 while the medial malleolus of the right foot is disposed within second ankle indentation 110. Nevertheless, it is also contemplated that in other implementations of the present disclosure the ankle indentations may not be symmetrically located relative to each other across the centerline of the insert/foot of the user, and instead may be positioned asymmetrically across the centerline to account for variations in the generally anatomy of the foot or for customization to the unique anatomy of specific individuals.

With foot 12 properly positioned relative to first ankle portion 104 and second ankle portion 108, first ankle portion 104 and second ankle portion 108 are secured by ankle fastener 128 of fastener assembly 126. In the implementation shown in FIG. 3, ankle fastener 128 can include an ankle strap 130 coupled to an exterior surface of first ankle cover portion 120. To secure ankle fastener 128, ankle strap 130 can be pulled across a lower shin 28 of patient 10 (or similar region of a calf 14 of patient 10) and secured to an exterior surface of second ankle portion 108 or at a similar location on an opposite side of lower shin 28. In the specific implementation shown, ankle fastener 128 can include a hook-and-loop style fastener to secure ankle strap 130. More specifically, ankle strap 130 ends in an ankle strap patch 132 including a first half of the hook-and-loop style fastener. When protective boot 100 is worn, ankle strap 130 extends across lower shin 28 and secures to an ankle cover patch 134 (shown in FIG. 1 and FIG. 4) attached to the exterior surface of second ankle portion 108 and which forms the second half of the hook-and-loop style fastener.

FIG. 4 illustrates protective boot 100 worn by patient 10 having their leg elevated to a horizontal position. Stated differently, FIG. 4 illustrates protective boot 100 on foot 12 of patient 10 following securing of plantar portion 112 using plantar fastener 136 and securing of each of first ankle portion 104 and second ankle portion 108 using ankle fastener 128.

As shown in FIG. 4, when worn, protective boot 100 can be bent at an approximately 90-degree angle such that the plantar portions 112 of protective boot 100 extends along sole 24 of the patient while first ankle portion 104 and second ankle portion 108 extend substantially parallel to calf 14. Stated differently, protective boot 100 forms a L-shape in which a first portion of protective boot 100 extends in a first (or vertical) direction along the sole 24 of patient 10 and a second portion of protective boot 100 extends in a second (or horizontal) direction substantially perpendicular to the first direction (e.g., in a direction parallel to the leg). As previously discussed in the context of FIG. 1, protective boot 100 may have varying dimensions to accommodate the anatomy of different patients. Nevertheless, in certain implementations, protective boot 100 may be configured such that, when worn, the first portion extending along the patient's sole is from and including about 9 inches to and including about 13 inches (e.g., 11.25 inches) while the second portion extending along the leg is from and including about 5.5 inches to and including about 8.5 inches (e.g., 7 inches).

Notably, in the specific implementation shown, plantar portion 112 terminates before toes 22 of patient, however, in other implementations plantar portion 112 may extend under or even beyond toes 22, particularly when additional toe support may be required.

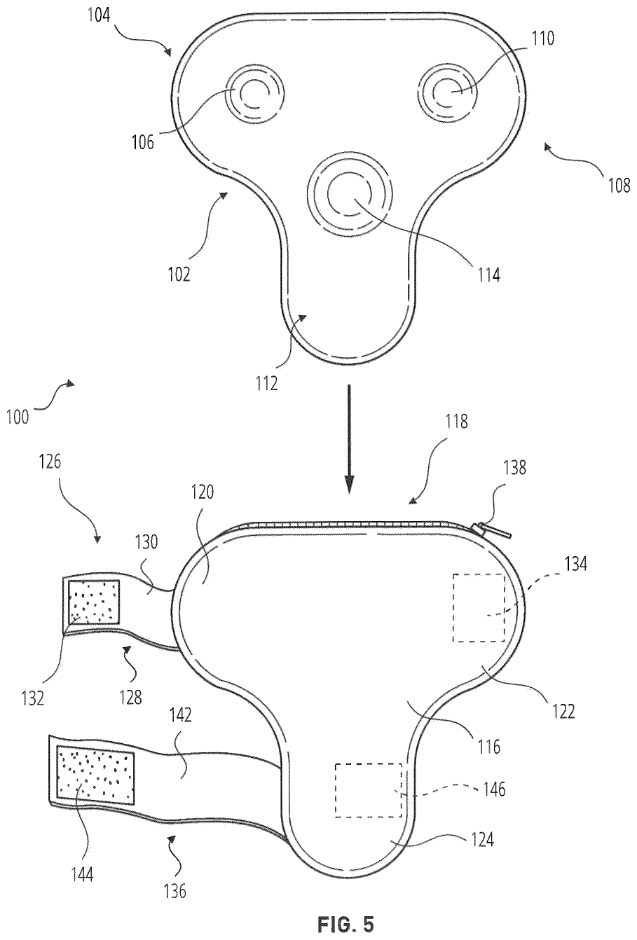

FIG. 5 is an exploded view of protective boot 100. As shown, each of insert 102 and cover 116 are generally T-shaped such that insert 102 may be disposed within and encapsulated by cover 116. For example, to assemble protective boot 100, insert 102 may be slid through an opening 118 disposed at the top of cover 116 and into cover 116. Following insertion of insert 102 into cover 116, opening 118 may be closed by securing closure 138. While closure 138 is shown in FIG. 5 as being a zipper, this disclosure contemplates that other closure types may be used. For example, cover 116 may include a flap extending along opening 118 securable by one or more hook-and-loop style fasteners, one or more clasps or buttons, one or more pieces/strips of reusable adhesive, one or more magnets, or other suitable closure mechanisms. Other locations for the opening 118, such as on the side or front of the cover, are also contemplated and considered to fall within the scope of the present disclosure.

In certain implementations cover 116 may be slightly oversized relative to insert 102 to accommodate insert 102. Alternatively, cover 116 may be formed from an elastic or semi-elastic material such that cover 116 can be stretched over insert 102 when protective boot 100 is assembled.

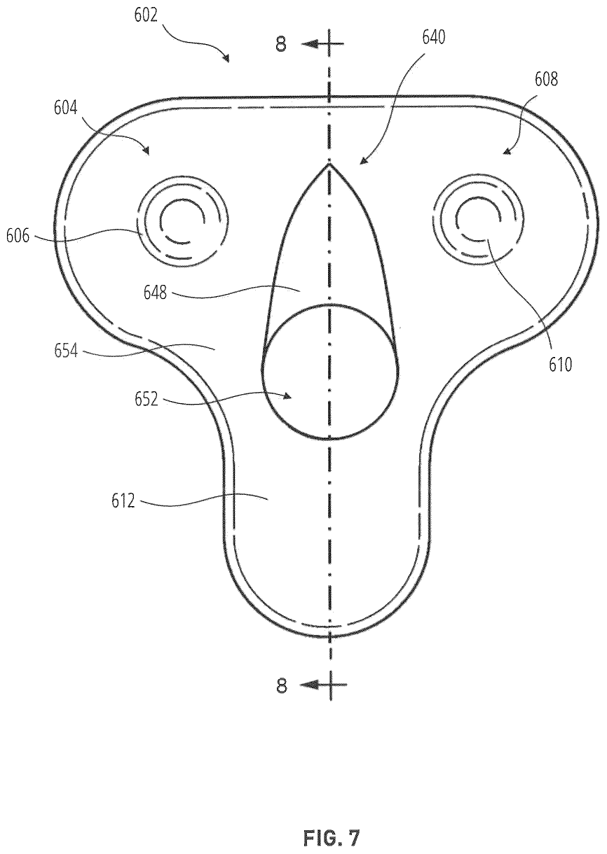

FIG. 6 is a plan view of a protective medical boot 600 according to another representative embodiment or implementation of the present disclosure. Like protective boot 100, protective boot 600 can include an insert 602 disposed within and enclosed by a cover 616, which each having the generally T-shaped construction described above. Insert 602 is illustrated as including each of a first ankle portion 604 which extends in a first direction and a second ankle portion 608 that generally extends equal and opposite first ankle portion 604. Insert 602 can further include a plantar portion 612 projecting perpendicular to each of first ankle portion 604 and second ankle portion 608. FIG. 7 and FIG. 8 illustrate insert 602 in further detail and, more specifically, in a plan view and cross-sectional view, respectively.

As with protective boot 100, first ankle portion 604 can include a first ankle indentation or pocket 606, and second ankle portion 608 can include a second ankle indentation or pocket 610 such that, when protective boot 600 is worn, the malleoli are received and protected within first ankle indentation 606 and second ankle indentation 610.

In contrast to protective boot 100, protective boot 600 can include a heel hole 614 instead of a heel indentation. Heel hole 614 can be defined by a cover heel hole 650 of cover 616 that aligns with an insert heel hole 652 of insert 602 when protective boot 600 is fully assembled. When protective boot 600 is worn by a patient, the heel of the patient can be received within heel hole 614 and remain exposed via the underside or backside of protective boot 600. Among other advantages, such a configuration can facilitate access to the heel to assess the condition of the heel while also improving air circulation around the heel.

In one aspect, a further distinction between protective boot 600 and protective boot 100 can be the inclusion of an Achilles support protrusion 648 in Achilles support portion or region 640, which can extend from heel hole 614 upwards between first ankle portion 604 and second ankle portion 608. In contrast to protective boot 100, in which Achilles support region 140 can be substantially flat and co-planar with a top surface of 102, Achilles support protrusion 648 can extend upward from a top surface 654 (shown in FIG. 7 and FIG. 8) of insert 102 and can have a curved shape that generally conforms to the Achilles tendon of the user, so as to provide direct support to the Achilles region. Notably, protective boot 100 may also be modified to include Achilles support protrusion 648. Stated differently, the inclusion of Achilles support protrusion 648 and/or heel hole 614 to the earlier embodiment or implementation are not mutually exclusive.

While Achilles support protrusion 648 is illustrated in the figures as being integrally formed with insert 602, in other implementations Achilles support protrusion 648 may instead be coupled to or otherwise integrated into cover 616. For example, Achilles support protrusion 648 may be in the form of a second insert and cover 616 may include a pocket (on either an inner or outer surface) shaped to receive the second insert. In yet other implementations, Achilles support protrusion 648 may alternatively be a thickened area of cover 616. For example, Achilles support protrusion 648 may correspond to a thickened area of cover 616 formed by coupling a patch to 616 or locally increasing layering of cover 616 in Achilles support region 640.

As with the dimensions of protective boot 100, dimensions of protective boot 600 may be varied to accommodate the particular anatomy and physiology of a patient/user. Protective boot 600 may be manufactured in a predetermined set of sizes intended collectively to accommodate a broad population. Alternatively, protective boot 600 may be custom sized and shape for particular patients. To the extent non-limiting example dimensions are provided above with respect to protective boot 100, such dimensions may similarly apply to implementations of protective boot 600.

As noted above, protective boot 600 can be distinct relative to protective boot 100 due to the including of heel hole 614 and Achilles support protrusion 648. While the dimensions of heel hole 614 may vary, in at least certain implementations, heel hole 614 may have a diameter from and including approximately 1.75 inches to and including about 3.5 inches. In other implementations, heel hole 614 may have a diameter from and including about 2 inches to and including about 2.5 inches, e.g., 2.25 inches.

FIG. 8 indicates certain dimensions relevant to Achilles support protrusion 648 and, more specifically, an Achilles support protrusion length 658 and an Achilles support protrusion height 660. As shown in FIG. 8, Achilles support protrusion length 658 generally corresponds to a distance between a center of insert heel hole 652 and an extent of Achilles support protrusion length 658. While Achilles support protrusion length 658 may vary, in at least certain implementations Achilles support protrusion length 658 is from and including about 4 inches to and including about 6 inches and, in certain specific implementations, may be approximately 5 inches. Achilles support protrusion height 660 is illustrated as a maximum height of Achilles support protrusion height 660 relative to a surface of insert 602 opposite Achilles support protrusion height 660. While Achilles support protrusion height 660 may vary, in at least certain implementations Achilles support protrusion height 660 may be from and including about 1.5 inches to and including 2.5 inches, e.g., 2.0 inches. Alternatively, Achilles support protrusion 648 may be defined, in part, based on an extension above top surface 654. For example, in certain implementations Achilles support protrusion 648 may extend from and including 0.25 inches to and including 1 inch above top surface 654, e.g., 0.5 inches.

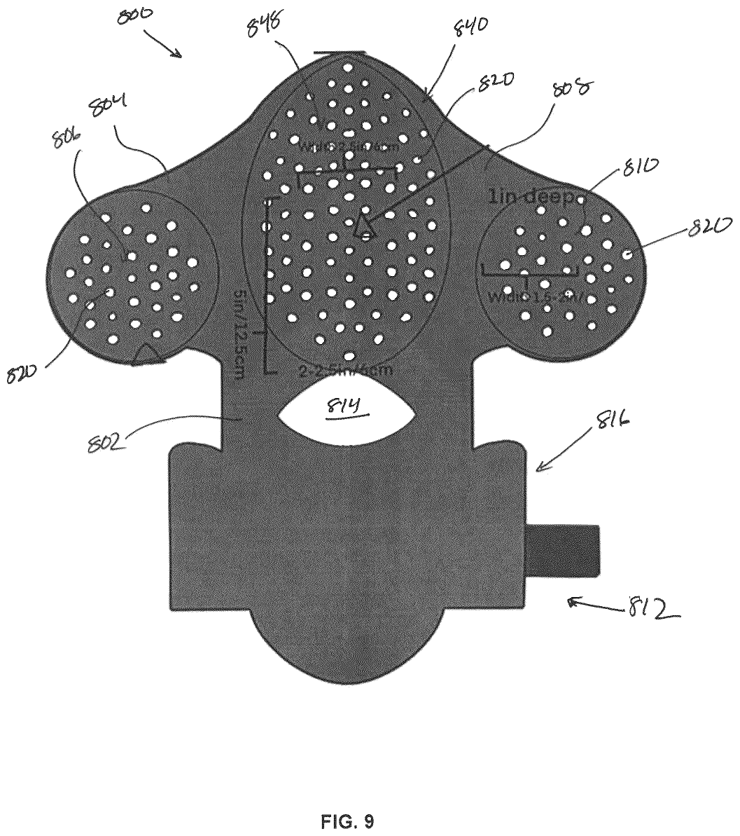

FIG. 9 is a plan view of a protective medical boot 800 according to another representative embodiment or implementation of the present disclosure. Similar to protective boot 100 and protective boot 600 describe above, protective boot 800 can include an insert 802 disposed within and enclosed by a cover 816, which each having a generally T-shaped construction. Insert 802 can also include each of a first ankle portion 804 which extends in a first direction and a second ankle portion 808 that generally extends equal and opposite first ankle portion 804. As shown in FIG. 9, in one aspect insert 802 can further include an Achilles support portion 840, which can extend from heel hole 814 upwards between first ankle portion 804 and second ankle portion 808. Insert 802 can further include a plantar portion 812 projecting perpendicular to each of first ankle portion 804 and second ankle portion 608, as well as a heel hole 814 located between the plantar portion 812 and the first and second ankle portions 804, 808 and the Achilles support portion 840. As with previous implementations, heel hole 814 can be defined by a cover heel hole 850 of cover 816 that aligns with an insert heel hole 852 of insert 802 when protective boot 800 is fully assembled.

As illustrated in FIG. 9, the shape of protective boot 800 can include an upper edge that is curved upwards above Achilles support portion 840, and which Achilles support portion 840 can include Achilles support protrusion 848 that is enlarged relative the size(s) of the first and second ankle indentations, or pockets, of the previous embodiments. It will be appreciated that, in one aspect, the enlarged Achilles support protrusion 848 and curved upper boundary of the Achilles support portion 840 may provide better support and comfort to the lower leg of a user.

As with previous embodiments, the ankle indentations or pockets 806, 810 of protective boot 800 can provide clearance as well as a supportive and protective covering for the malleoli of the user by better conforming to the anatomy of the foot. Moreover, each of the ankle indentations or pockets 806, 810 of protective boot 800 can also include a plurality of small openings or perforations 820 extending through the thickness of the insert 102 that can provide for increased airflow and circulation of air around the ankle portion of the foot, which can provide for improved cooling (or heating) of the foot to increase the comfort level of the user, as well as removal of excess moisture to promote the healing of any pressure and/or abrasive sores located in those areas. As shown in FIG. 9, perforations 820 can extend through both insert 802 and cover 816 to provide clear pathways for airflow. In other aspects, however, it may be sufficient that perforations 820 are only be formed in insert 802, with the air and any excess moisture easily passing through the porous fabric of the cover 816.

In addition to having the curved shape that generally conforms to the Achilles tendon of the user, so as to provide direct support to the Achilles region, the enlarged Achilles support region 840 of protective boot 800 can also be formed with the plurality of small openings or perforations 820 extending through the increased thickness of the Achilles support protrusion 848, for the same reasons and benefits provided to the ankle indentations or pockets 806, 810. While the size and shape of perforations 820 are shown as being uniform across the insert 802, it will be appreciated that the size, shape, number and placement of perforations 820 may vary or differ from those shown in the drawings.

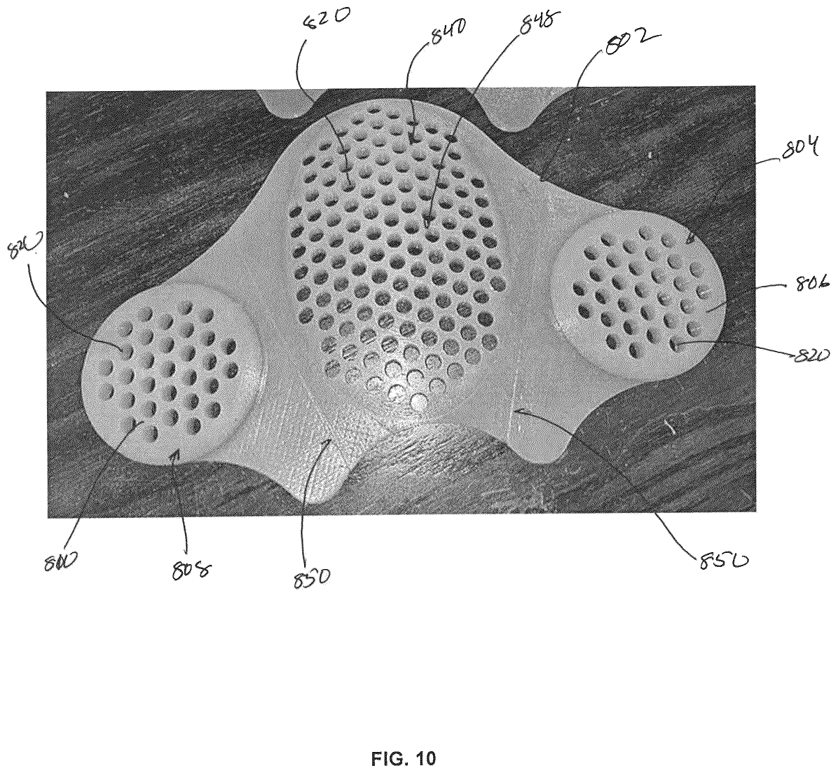

FIG. 10 is a backside perspective view of insert 802 showing ankle pockets 806, 810 and Achilles support protrusion 848 in further detail. As can be seen in the figure, insert 802 can be molded or formed from a shaped layer of solid gel or gel-like flexible material having an average thickness that is substantially less than the thickness of the ankle pockets 806, 810 and the Achilles support protrusion 848 which, in one aspect, can serve to make the overall structure of insert 802 more flexible. Also shown are diagonally-extending creases 850 between the ankle pockets 806, 810 and the Achilles support protrusion 848, which creases can be pre-formed into the insert to promote the folding of protective boot 800, while maintaining a flexible panel-like construction for each portion of the insert 802, when positioning protective boot 800 around the foot of a user. Other variations in the thickness, shape, and construction of the insert 802 are also possible and considered to fall within the scope of the present disclosure.

Aspects of the present disclosure have been described herein in terms of preferred embodiments and methodologies considered by the inventor to represent the best mode of carrying out the invention. It will be understood by the skilled artisan, however, that a wide range of additions, deletions, and modifications, both subtle and gross, may be made to the illustrated and exemplary embodiments of protective boots without departing from the spirit and scope of the invention. These and other revisions might be made by those of skill in the art without departing from the spirit and scope of the invention that is constrained only by the following claims.

What is claimed is:

1. A protective boot for shielding and promoting the healing of a foot of a user, the protective boot comprising:
an insert including:
a first ankle portion including a first ankle indentation shaped and positioned to receive a medial malleolus of a user when the protective boot is worn;
a second ankle portion including a second ankle indentation shaped and positioned to receive a lateral malleolus of the user when the protective boot is worn; and
a plantar portion including a heel indentation, the heel indentation shaped and positioned to receive a heel of the user when the protective boot is worn; and
a cover conforming to and enclosing the insert, the cover including:
an opening shaped to receive the insert; and
a fastener assembly for securing the protective boot around the foot of the user.

2. The protective boot of claim 1, wherein the insert is formed from a solid gel or gel-like flexible material.

3. The protective boot of claim 1, wherein the insert is formed from a silicone rubber gel.

4. The protective boot of claim 1, further comprising a plurality of protrusions formed completely through a thickness of the insert at the first ankle indentation and at the second ankle indentation.

5. The protective boot of claim 1, wherein the insert has a unitary structure.

6. The protective boot of claim 1, wherein the insert includes at least two distinct portions.

7. The protective boot of claim 1, wherein, when unworn, the insert has a substantially planar shape.

8. The protective boot of claim 1,
wherein the cover includes a first ankle cover portion corresponding to the first ankle portion of the insert, a second ankle cover portion corresponding to the second ankle portion of the insert, and a plantar cover portion corresponding to the plantar portion of the insert, and
wherein the fastener assembly includes an ankle fastener configured to extend between the first ankle cover portion and the second ankle cover portion, such that, when the protective boot is worn by the user and the ankle fastener is engaged, the first ankle portion and the second ankle portion of the insert conform to the ankle of the user.

9. The protective boot of claim 8, wherein the ankle fastener further comprises a hook-and-loop style fastener including:
a strap attached to an exterior surface of the first ankle cover portion and terminating in a first patch including a first surface of the hook-and-loop style fastener; and
a second patch including a second surface of the hook-and-loop style fastener coupled to the second ankle cover portion.

10. The protective boot of claim 8, wherein the fastener assembly further includes a plantar fastener configured such that, when the protective boot is worn by the user, the plantar fastener extends across an instep of the user.

11. The protective boot of claim 10, wherein the plantar fastener further comprises a hook-and-loop style fastener including:
a strap attached to an exterior surface of the plantar portion and terminating in a first patch including a first surface of the hook-and-loop style fastener; and
a second patch coupled to the exterior surface of the plantar portion, the second patch including a second surface of the hook-and-loop style fastener.

12. The protective boot of claim 1, wherein the cover is formed from a moisture-wicking material.

13. The protective boot of claim 12, wherein the moisture-wicking material is one of polyester, polypropylene, nylon, micromodal rayon, and bamboo.

14. The protective boot of claim 1, wherein the cover is formed from a quick-drying material.

15. The protective boot of claim 14, wherein the quick-drying material is one of polyester and a polyester-cotton blend.

16. The protective boot of claim 1, further comprising an Achilles support protrusion extending from the heel indentation between the first ankle portion and the second ankle portion.

17. The protective boot of claim 16, wherein the Achilles support protrusion is integrally formed with the insert.

18. The protective boot of claim 16, wherein the Achilles support protrusion includes a plurality of perforations formed completely through a thickness thereof.

19. The protective boot of claim 1, wherein the heel indentation extends through a thickness of the insert such that the heel indentation forms a heel hole.
